(19) United States
(12) Patent Application Publication
(10) Pub. No.: US 2016/0102299 A1
(43) Pub. Date: Apr. 14, 2016

(54) POLYPEPTIDES ENCODING MUTATED MANNANASES WITH IMPROVED CATALYTIC EFFICIENCY

(71) Applicants: INSTITUT NATIONAL DE RECHERCHE AGRONOMIQUE (INRA), Paris Cedex 7 (FR); IFP ÉNERGIES NOUVELLES (IFPEN), Rueil-Malmaison Cedex (FR); AGRO-INDUSTRIE RECHERCHES ET DEVELOPPEMENTS (ARD), Pomacle (FR)

(72) Inventors: Jean-Guy BERRIN, Marseille (FR); Marie COUTURIER, Marseille (FR)

(21) Appl. No.: 14/771,912

(22) PCT Filed: Feb. 27, 2014
(86) PCT No.: PCT/EP2014/000517

§ 371 Date: Sep. 1, 2015

(30) Foreign Application Priority Data
Mar. 1, 2013 (FR) 13/00467

(51) Int. Cl. C12N 9/24; C12P 19/02; C12P 19/14

(52) U.S. Cl. CPC C12N 9/2488; C12P 19/14; C12P 19/02; C12Y 302/01

(57) ABSTRACT

A polypeptide which is a mutated mannanase, whose sequence is derived from a native mannanase of the filamentous ascomycete coprophile fungus Podospora anserina, and being characterised by an increased catalyst efficiency of at least 25%. Also, a polynucleotide encoding the polypeptide, a vector including the polynucleotide, a host cell including the vector or the polynucleotide, and a composition including the polypeptide, the polynucleotide, the vector or the host cell. Methods of using the polypeptide, the polynucleotide, the vector, the host cell or the composition of the invention for degradation of compounds including mannans, in particular lignocellulosic biomass, as well as various possible uses of the polypeptide, the polynucleotide, the vector, the host cell or the composition.

The present international application claims priority of French patent application FR 13/00467 filed on Mar. 1, 2013.

### FIELD OF THE INVENTION

The invention relates to polypeptides encoding mannanases mutants having improved enzyme performance. The invention also provides polynucleotides encoding such polypeptides, vectors and host cells comprising such polynucleotides, as well as compositions comprising such polypeptide (s), polynucleotide (s), vector (s) or host cell (s).

Finally, the invention covers the various uses of such polypeptides, polynucleotides, vectors, host cells or compositions.

### BACKGROUND ART

The conversion of biomass, particularly lignocellulosic biomass, into simple sugars is widely studied because it is a prerequisite to the fermentation of the degradation products thereof for the production of bioethanol or various industrial products.

Saccharification, that is to say the enzymatic hydrolysis of components of lignoccllulosic biomass, is today one of the main stumbling blocks to the biological refinery process: the resistance of the plant cell walls leads to the need to use a large quantity of enzymes to hydrolyse the lignocellulosic biomass into fermentable sugars. The conversion of lignocellulosic biomass is therefore today a high cost reaction.

The ascomycete fungus Trichoderma reesei is now one of the most used industrial fungi worldwide. It is in fact capable of producing an enzyme cocktail rich in cellulases used for degradation of lignocellulosic biomass.

However, the considerable costs associated with the use of such enzyme cocktails for the degradation of the lignocellulosic biomass constitute a barrier to the use of this renewable resource.

It is therefore necessary to improve existing enzyme cocktails to increase their effectiveness and efficiency, to reduce the cost price of biomass conversion.

Lignocellulose is the most abundant component of biomass, comprising about fifty percent of plant material produced by photosynthesis and representing the most important renewable biological resource in the ground. It contains mainly three types of polymers: cellulose, hemicellulose and lignin. Cellulose represents about 45% of the dry weight of lignocellulose. It is a linear polymer composed of D-glucose linked by β1,4-glycosidic bond, forming long chains linked together by noncovalent bonds. Hemicellulose is formed of heteropolymers representing 15 to 35% of the biomass, and contain pentoses, such as β-D-xylose and L-α-arabinose, hexoses, such as β-D-mannose, the β-D-glucose or α-D-galactose, or uronic acids. Lignin is composed of phenylpropane units bonded together by various types of bonds. It binds to the cellulose and hemicellulose and forms a physical barrier to protect plants, more specifically plant cells.

Hemicellulose refers to a diverse set of non-crystalline carbohydrate polymers, among which mannans are a major component, including softwood. Mannans are a family of complex sugars comprising a structure of residues of D-mannose, called mannan, or a combination of residues of β1,4-D-mannose and β1,4-D-glucose, called glucomannan. Each of the two structures can be complemented with side chains of galactose, and then forms a saccharide polymer called galactomannan and galactoglucomannan respectively.

Mannans, broadly speaking, are hydrolysed through coordinated action of different types of glycoside hydrolases including in particular the mannanases. Mannanases are hence enzymes necessary for the conversion of lignocellulosic biomass.

Two mannanases derived from coprophile Podospora anserina fungus has recently been studied and identified as factors increasing the efficiency of the enzyme cocktail to Trichoderma reesei for the degradation of lignocellulosic biomass (Couturier et al, Applied and Environmental Microbiology, January 2011, 77(1):23, pages 237-246).

This study is a way of improving existing enzyme cocktails for degradation of lignocellulosic biomass. Additional studies could allow the emergence of new enzymes or new tools to improve their effectiveness.

### SUMMARY OF THE INVENTION

Seeking to improve the existing means of degradation of the lignocellulosic biomass, the inventors have shown that the effectiveness of prior enzyme cocktails currently available for lignocellulose degradation, such as those derived from Trichoderma reesei secretome, can be enhanced by the supplementation of these with additional enzymes, including mannanases Man5A and Man26A from the ascomycete Podospora anserina fungus (Couturier et al., 2011).

Following these results, the inventors have sought to further increase the efficiency of an enzymatic cocktail of supplemented Trichoderma reesei. They have thus sought to develop variants of the Man5A and Man26A mannanases, having improved enzyme activities compared with the native enzymes.

The invention thus relates to a polypeptide consisting of a mutated mannanase, having a sequence derived from a native mannanase of the filamentous ascomycete coprophile fungus Podospora anserina, and being characterized by an increased catalyst efficiency of at least 25% compared to the catalytic efficiency of this native mannanase.

More particularly, said polypeptide has been derived from the mannanase Man5A or Man26A of Podospora anserina and is defined by one of the sequences SEQ ID NO: 3 to 14, and differs by at least one amino acid of SEQ ID NO: 1 or 11.

The invention also relates to a polynucleotide encoding such a peptide, a vector comprising such a polynucleotide and a host cell comprising such a polynucleotide or such a vector.

The invention further relates to a composition comprising a polypeptide, a polynucleotide, a vector or a host cell of the invention.

The invention finally relates to the use of a polypeptide, a polynucleotide, a vector, a host cell or a composition of the invention for degradation of compounds comprising mannans, in particular, for the degradation of the lignocellulosic biomass.

### DETAILED DESCRIPTION OF THE PRESENT INVENTION

Seeking to improve the efficiency of the enzyme cocktails used for degradation of the lignocellulosic biomass, the inventors have developed mannanases mutants from the filamentous ascomycete coprophile fungus Podospora anserina, said mutant exhibiting improved catalytic efficiency of at least 25% with respect to the native enzymes.

The invention thus relates to a polypeptide consisting of a mutated mannanase, having a sequence derived from a native mannanase of the filamentous ascomycete coprophile fungus Podospora anserina, and being characterized by an increased catalyst efficiency of at least 25% compared to the catalytic efficiency of this native mannanase.

The term “mannanase” refers to a family of enzymes capable of hydrolysing polyose chains composed of mannose (called mannans, mannopolymers or polymannoses).

By “mutated mannanase” is meant a mannanase defined by a sequence derived from a native mannanase and whose sequence comprises at least one mutation with respect to the sequence of this native mannanase.

The term “mutation” refers to the substitution, the replacement, the insertion or the deletion of one or more amino acids in a reference protein sequence.

A mutated mannanase of the invention is characterised by an increased catalyst efficiency of at least 25% compared to the catalytic efficiency of the native mannanase.

The catalytic efficiency of an enzymatic reaction, which is associated with a given enzyme, is well known to those skilled in the art and is commonly determined by measuring the k_{cat}/K_{M} ratio, where k_{cat} is the catalytic constant, corresponding to the number of moles of product formed per second and per mol of enzyme, and where K_{M} is the Michaelis constant, characteristic of the enzyme tested.

Preferably, the catalytic efficiency of the polypeptides of the invention is measured on the hydrolysis reaction of the galactomannan and compared to that of native mannanase. The hydrolysis reactions of mannooligosaccharides such as mannopentaose and mannohexaose may also be used. Such reactions and measurements are described in Berrin et al, 2007 (Appl Microbiol Biotechnol., 74(5):1001).

In a particular embodiment of the invention, the polypeptide has been derived from Man5A mannanase of Podospora anserina.

By “Man5A” is meant Man5A mannanase of Podospora anserina, as defined by the protein sequence SEQ ID NO: 1. This mannanase is encoded by the nucleic sequence SEQ ID NO: 2.

SEQ ID No
LPQAQGGGAA ASAKVSGTRF VIDGKTGYFA GTNSYWIGFL
TNNRDVDTTL DHIASSGLKI LRVWGFNDVN NQPSGNTVWF
QRLASSGSQI NTGPNGLQRL DYLVRSAETR GIKLIIALVN
YWDDFGGMKA YVNAFGGTKE SWYTNARAQE QYKRYIQAVV
SRYVNSPAIF AWELANEPRC KGCNTNVIFN WATQISDYIR
SLDKDHLITL GDEGFGLPGQ TTYPYQYGEG TDFVKNLQIK
NLDFGTFHMY PGHWGVPTSF GPGWIKDHAA ACRAAGKPCL
LEEYGYESDR CNVQKGWQQA SRELSRDGMS GDLFWQWGDQ
LSTGQTHNDG FTIYYGSSLA TCLVTDHVRA INALPA
SEQ ID NO: 2
TCCCCCAAGCACAAGGTGGAGGAGCAGCCGCCTCAGCCAAAGTCAGCGGC
ACCCGCTTCGTGATCGACGGCAAAACCGGCTACTTTGCAGGAACAAACTC
CTACTGGATTGGCTTCCTGACCAACAACAGAGATGTCGACACAACCTTGG
ACCACATCGCCTCCTCGGGCCTCAAAATCCTCCGCGTCTGGGGCTTCAAC
GACGTGAACAACCAACCATCCGGTAACACCGTCTGGTTCCAACGCCTCGC
CTCCTCAGGCTCCCAAATCAACACCGGCCCCAACGGCCTCCAACGCCTCG
ACTACCTCGTCAGATCAGCCGAAACCCGCGGCATCAAGCTCATCATCGCG
CTGGTCAACTACTGGGATGACTTTGGCGGCATGAAAGCCTACGTCAACGC
CTTTGGAGGCACAAAAGAATCCTGGTACACCAACGCCCGCGCTCAGGAGC
AGTACAAGCGTTACATCCAGGCTGTCGTCTCGCGATATGTCAACAGCCCC
GCAATCTTTGCGTGGGAACTTGCCAACGAGCCCAGGTGCAAGGGGTGCAA
CACGAATGTTATTTTCAACTGGGCGACGCAGATTTCAGATTATATCCGGA
GCTTGGATAAGGATCATTTGATCACCCTTGGGGATGAGGGGTTTGGGTTG
CCGGGGCAGACGACGTATCCGTATCAGTATGGGGAGGGGACCGACTTCGT
CAAGAATCTGCAGATTAAGAATCTGGACTTTGGGACGTTTCATATGTATC
CTGGTCATTGGGGGGTGCCGACGAGTTTTGGTCCAGGGTGGATTAAGGAT
CATGCGGCGGCTTGCAGGGCGGCGGGGAAGCCGTGTTTGTTGGAGGAGTA
TGGGTATGAGAGTGATAGGTGTAATGTGCAGAAGGGCTGGCAGCAGGCGT
CGAGGGAGCTGAGCAGGGATGGGATGAGTGGTGATTTGTTTTGGCAATGG
GGCGATCAGTTGAGTACTGGGCAGACACATAATGATGGGTTCACGATTTA
TTATGGTTCTTCGTTGGCTACTTGCTTGGTTACTGACCATGTGAGGGCTA
TCAATGCTCTCCCGGCGTAG

According to the invention, the polypeptide of the invention is defined by the sequence SEQ ID NO: 3

SEQ ID NO: 3
LPQAQGGGAA ASAKVSGTRF VIDGKTGYFA GTNSYX³⁶IGFL
TNNRDVDTTL DHIASSGLKI LRVWGFNDVN NQPSGNTVWF
QRLASSGSQI NTGPNGLQRL DYLVRSAETR GIKLIIALVN
YWDDFGGMKA YVNAFGGTX¹³⁹E SWYTNARAQE QYKRYIQAVV
SRYVNSPAIF AWELANEPRC KGCNTNVIFN WATQX¹⁹⁵SDYIR
SLDKDHLITL GDEGFGLPGQ TTX²²³PYQYGEG TDFVKNLQIK
NLDFGTFHMY PGHWGX²⁵⁶PTSF GPGWIKDHAA ACRAAX²⁷⁶KPCL
LEEYGYESDR CNVQKGWQQA SRELSRDGMS X³¹¹DLFWX³¹⁶WGDQ
LSTGQTHNDG FTIYYGSSLA TCLVTDHVRA INALPA
Where:
the residue at position 36 is a tryptophan or
arginine (X36 = W or R)
the residue at position 139 is a tryptophan or
arginine (X139 = W or R)
the residue at position 195 is an isoleucine or
threonine (X195 = I or T),
the residue at position 223 is a tyrosine or
histidine (X223 = Y or H),
the residue at position 256 is a valine, leucine
or alanine (X256 = V, L or A),
the residue at position 276 is a glycine or a
valine (X276 = G or V),
the residue at position 311 is a glycine or a
serine (X311 = G or S),
and
the residue at position 316 is a glutamine or
histidine (X316 = Q or H),

SEQ ID NO: 3 differs by at least one amino acid from SEQ ID NO: 1.

In a preferred embodiment, the polypeptide of the invention is defined by the sequence SEQ ID NO: 4

SEQ ID NO: 4
LPQAQGGGAA ASAKVSGTRF VIDGKTGYFA GTNSYWIGFL
TNNRDVDTTL DHIASSGLKI LRVWGFNDVN NQPSGNTVWF
QRLASSGSQI NTGPNGLQRL DYLVRSAETR GIKLIIALVN
YWDDFGGMKA YVNAFGGTKE SWYTNARAQE QYKRYIQAVV
SRYVNSPAIF AWELANEPRC KGCNTNVIFN WATQISDYIR
SLDKDHLITL GDEGFGLPGQ TTYPYQYGEG TDFVKNLQIK
NLDFGTFHMY PGHWGVPTSF GPGWIKDHAA ACRAAGKPCL
LEEYGYESDR CNVQKGWQQA SRELSRDGMS SDLFWQWGDQ
LSTGQTHNDG FTIYYGSSLA TCLVTDHVRA INALPA

The catalytic effectiveness of the polypeptide defined by SEQ ID NO: 4 (G311S mutant) is increased by a factor of 8 (800% increase) compared to that of native mannanase Man5A (SEQ ID NO: 1) on the hydrolysis reaction of galactomannan.

In a preferred embodiment, the polypeptide of the invention is defined by the sequence SEQ ID NO: 5

SEQ ID NO: 5
LPQAQGGGAA ASAKVSGTRF VIDGKTGYFA GTNSYWIGFL
TNNRDVDTTL DHIASSGLKI LRVWGFNDVN NQPSGNTVWF
QRLASSGSQI NTGPNGLQRL DYLVRSAETR GIKLIIALVN
YWDDFGGMKA YVNAFGGTX¹³⁹E SWYTNARAQE QYKRYIQAVV
SRYVNSPAIF AWELANEPRC KGCNTNVIFN WATQISDYIR
SLDKDHLITL GDEGFGLPGQ TTX²²³PYQYGEG TDFVKNLQIK
NLDFGTFHMY PGHWGVPTSF GPGWIKDHAA ACRAAGKPCL
LEEYGYESDR CNVQKGWQQA SRELSRDGMS GDLFWQWGDQ
LSTGQTHNDG FTIYYGSSLA TCLVTDHVRA INALPA
Where:
the residue at position 139 is a lysine or an
arginine (X139 = K or R),
and
the residue at position 223 is a tyrosine or a
histidine (X223 = Y or H).

SEQ ID NO: 5 differs by at least one amino acid from SEQ ID NO: 1.

More preferably, the polypeptide of the invention is defined by the sequence SEQ ID NO: 6.

SEQ ID NO: 6
LPQAQGGGAA ASAKVSGTRF VIDGKTGYFA GTNSYWIGFL
TNNRDVDTTL DHIASSGLKI LRVWGFNDVN NQPSGNTVWF
QRLASSGSQI NTGPNGLQRL DYLVRSAETR GIKLIIALVN
YWDDFGGMKA YVNAFGGTRE SWYTNARAQE QYKRYIQAVV
SRYVNSPAIF AWELANEPRC KGCNTNVIFN WATQISDYIR
SLDKDHLITL GDEGFGLPGQ TTHPYQYGEG TDFVKNLQIK
NLDFGTFHMY PGHWGVPTSF GPGWIKDHAA ACRAAGKPCL
LEEYGYESDR CNVQKGWQQA SRELSRDGMS GDLFWQWGDQ
LSTGQTHNDG FTIYYGSSLA TCLVTDHVRA INALPA

The catalytic effectiveness of the polypeptide defined by SEQ ID NO: 6 (K139R/Y223H mutant) is increased by a factor of 1.7 (70% increase) compared to that of native mannanase Man5A (SEQ ID NO: 1) on the hydrolysis reaction of galactomannan.

In another preferred embodiment, the polypeptide of the invention is defined by the sequence SEQ ID NO: 7.

SEQ ID NO: 7
LPQAQGGGAA ASAKVSGTRF VIDGKTGYFA GTNSYWIGFL
TNNRDVDTTL DHIASSGLKI LRVWGFNDVN NQPSGNTVWF
QRLASSGSQI NTGPNGLQRL DYLVRSAETR GIKLIIALVN
YWDDFGGMKA YVNAFGGTKE SWYTNARAQE QYKRYIQAVV
SRYVNSPAIF AWELANEPRC KGCNTNVIFN WATQISDYIR
SLDKDHLITL GDEGFGLPGQ TTYPYQYGEG TDFVKNLQIK
NLDFGTFHMY PGHWGX²⁵⁶PTSF GPGWIKDHAA ACRAAX²⁷⁶KPCL
LEEYGYESDR CNVQKGWQQA SRELSRDGMS GDLFWX³¹⁶WGDQ
LSTGQTHNDG FTIYYGSSLA TCLVTDHVRA INALPA
Where:
the residue at position 256 is a valine, a
leucine or an alanine (X256 = V, L or A),
the residue at position 276 is a glycine or a
valine (X276 = G or V),
the residue at position 316 is a glutamine or
histidine (X316 = Q or H),

SEQ ID NO: 7 differs by at least one amino acid from SEQ ID NO: 1.

More preferably, the polypeptide of the invention is defined by the sequence SEQ ID NO: 8.

SEQ ID NO: 8
LPQAQGGGAA ASAKVSGTRF VIDGKTGYFA GTNSYWIGFL
TNNRDVDTTL DHIASSGLKI LRVWGFNDVN NQPSGNTVWF
QRLASSGSQI NTGPNGLQRL DYLVRSAETR GIKLIIALVN
YWDDFGGMKA YVNAFGGTKE SWYTNARAQE QYKRYIQAVV
SRYVNSPAIF AWELANEPRC KGCNTNVIFN WATQISDYIR
SLDKDHLITL GDEGFGLPGQ TTYPYQYGEG TDFVKNLQIK
NLDFGTFHMY PGHWGLPTSF GPGWIKDHAA ACRAAVKPCL
LEEYGYESDR CNVQKGWQQA SRELSRDGMS GDLFWHWGDQ
LSTGQTHNDG FTIYYGSSLA TCLVTDHVRA INALPA

The catalytic effectiveness of the polypeptide defined by SEQ ID NO: 8 (V256L/G276V/Q316H mutant) is increased by a factor of 1.3 (30% increase) compared to that of native mannanase Man5A (SEQ ID NO: 1) on the hydrolysis reaction of galactomannan.

In another preferred embodiment, the polypeptide of the invention is defined by the sequence SEQ ID NO: 9.

SEQ ID NO: 9
LPQAQGGGAA ASAKVSGTRF VIDGKTGYFA GTNSYX³⁶IGFL
TNNRDVDTTL DHIASSGLKI LRVWGFNDVN NQPSGNTVWF
QRLASSGSQI NTGPNGLQRL DYLVRSAETR GIKLIIALVN
YWDDFGGMKA YVNAFGGTKE SWYTNARAQE QYKRYIQAVV
SRYVNSPAIF AWELANEPRC KGCNTNVIFN WATQX¹⁹⁵SDYIR
SLDKDHLITL GDEGFGLPGQ TTYPYQYGEG TDFVKNLQIK
NLDFGTFHMY PGHWGX²⁵⁶PTSF GPGWIKDHAA ACRAAGKPCL
LEEYGYESDR CNVQKGWQQA SRELSRDGMS GDLFWQWGDQ
LSTGQTHNDG FTIYYGSSLA TCLVTDHVRA INALPA
Where:
the residue at position 36 is a tryptophan or
an arginine (X36 = W or R),
the residue at position 195 is an isoleucine
or a threonine (X195 = I or T),
the residue at position 256 is a valine, a
leucine or an alanine (X256 = V, L or A),

SEQ ID NO: 9 differs by at least one amino acid from SEQ ID NO: 1.

More preferably, the polypeptide of the invention is defined by the sequence SEQ ID NO: 10.

SEQ ID NO: 10
LPQAQGGGAA ASAKVSGTRF VIDGKTGYFA GTNSYRIGFL
TNNRDVDTTL DHIASSGLKI LRVWGFNDVN NQPSGNTVWF
QRLASSGSQI NTGPNGLQRL DYLVRSAETR GIKLIIALVN
YWDDFGGMKA YVNAFGGTKE SWYTNARAQE QYKRYIQAVV
SRYVNSPAIF AWELANEPRC KGCNTNVIFN WATQTSDYIR
SLDKDHLITL GDEGFGLPGQ TTYPYQYGEG TDFVKNLQIK
NLDFGTFHMY PGHWGAPTSF GPGWIKDHAA ACRAAGKPCL
LEEYGYESDR CNVQKGWQQA SRELSRDGMS GDLFWQWGDQ
LSTGQTHNDG FTIYYGSSLA TCLVTDHVRA INALPA

The catalytic effectiveness of the polypeptide defined by SEQ ID NO: 10 (W36R/I195TN256A mutant) is increased by a factor of 1.78 (78% increase) compared to that of native mannanase Man5A (SEQ ID NO: 1) on the hydrolysis reaction of galactomannan.

In another particular embodiment of the invention, the mutated mannanase of the invention has been derived from Man26A mannanase of Podospora anserina.

By “Man26A” is meant Man26A mannanase of Podospora anserina, as defined by the protein sequence SEQ ID NO: 11 and the nucleic sequence SEQ ID NO: 12.

SEQ ID No
KPCKPRDGPV TYEAEDAILT GTTVDTAQVG YTGRGYVTGF
DEGSDKITFQ ISSATTKLYD LSIRYAAIYG DKRTNVVLNN
GAVSEVFFPA GDSFTSVAAG QVLLNAGQNT IDIVNNWGWY
LIDSITLTPS APRPPHDINP NLNNPNADTN AKKLYSYLRS
VYGNKIISGQ QELHHAEWIR QQTGKTPALV AVDLMDYSPS
RVERGTTSHA VEDAIAHHNA GGIVSVLWHW NAPVGLYDTE
ENKWWSGFYT RATDFDIAAT LANPQGANYT LLIRDIDAIA
VQLKRLEAAG VPVLWRPLHE AEGGWFWWGA KGPEPAKQLW
DILYERLTVH HGLDNLIWVW NSILEDWYPG DDTVDILSAD
VYAQGNGPMS TQYNELIALG RDKKMIAAAE VGAAPLPGLL
QAYQANWLWF AVWGDDFINN PSWNTVAVLN EIYNSDYVLT
LDEIQGWRS
SEQ ID NO: 12
AAGCCTTGTAAGCCTCGTGATGGCCCCGTGACCTACGAGGCCGAAGATGC
CATCCTCACCGGCACCACCGTCGACACTGCTCAGGTAGGCTATACCGGCC
GTGGCTACGTCACCGGCTTCGACGAGGGCTCCGACAAGATCACCTTCCAG
ATCAGCTCCGCCACCACCAAGCTCTACGACCTCTCCATCCGGTACGCCGC
CATCTACGGTGACAAGCGAACCAACGTCGTCCTCAACAACGGTGCCGTCA
GCGAGGTCTTCTTCCCCGCCGGTGACTCTTTTACTTCTGTCGCCGCCGGC
CAGGTCCTCCTCAACGCTGGACAAAACACCATCGACATCGTCAACAACTG
GGGATGGTACCTCATCGACTCCATCACCCTCACCCCCTCCGCCCCTCGCC
CCCCCCACGACATCAACCCCAACCTCAACAACCCCAACGCCGACACCAAC
GCCAAGAAGCTCTACTCCTACCTCCGCTCTGTCTACGGCAACAAGATCAT
CTCTGGCCAGCAGGAGCTCCACCACGCCGAGTGGATCAGACAGCAAACCG
GCAAGACTCCCGCGCTGGTGGCTGTCGATCTGATGGATTACTCCCCCTCC
CGCGTCGAGCGTGGCACCACCAGCCATGCCGTCGAGGACGCCATCGCCCA
CCACAACGCAGGCGGTATCGTTTCTGTCCTCTGGCACTGGAACGCTCCCG
TCGGTCTGTATGACACCGAAGAGAACAAGTGGTGGTCCGGCTTCTACACT
CGGGCTACCGACTTTGACATTGCCGCCACGTTGGCCAACCCCCAGGGTGC
GAACTACACTCTTCTCATCAGGGACATTGACGCGATTGCTGTCCAGCTCA
AGAGGCTGGAGGCTGCTGGTGTTCCGGTCTTGTGGAGACCTCTTCACGAG
GCGGAGGGTGGTTGGTTCTGGTGGGGAGCCAAGGGGCCAGAGCCGGCGAA
GCAGCTTTGGGATATCTTGTATGAGCGTCTGACGGTGCACCATGGTTTGG
ATAATTTGATTTGGGTGTGGAATTCGATTTTGGAGGATTGGTATCCGGGT
GATGATACGGTTGATATCTTGTCGGCCGATGTGTATGCGCAGGGTAATGG
GCCCATGTCGACTCAGTACAATGAGTTGATCGCCCTCGGCAGGGACAAGA
AGATGATTGCTGCTGCAGAGGTTGGCGCTGCCCCTTTGCCCGGCTTGTTG
CAGGCTTACCAGGCCAACTGGCTGTGGTTTGCTGTCTGGGGTGATGACTT
TATCAACAACCCCAGCTGGAACACGGTTGCTGTTCTCAACGAGATCTACA
ACAGCGACTATGTGTTGACGCTGGATGAGATTCAGGGGTGGAGGAGTTAG

In a more particular embodiment, the mutated mannanase of the invention derived from Man26A is defined by SEQ ID NO: 13.

SEQ ID NO: 13
KPCKPRDGPV TYEAEDAILT GTTVDTAQVG YTGRGYVTGF
DEGSDKITFQ ISSATTKLYD LSIRYAAIYG DKRTNVVLNN
GAVSEVFFPA GDSFTSVAAG QVLLNAGQNT IDIVNNWGWY
LIDSITLTPS APRPPHDINX¹⁴⁰ NLNNPNADTN AKKLYSYLRS
VYGNKIISGQ QELHHAEWIR QQTGKTPALV AVDLMDYSPS
RVERGTTSHA VEDAIAHHNA GGIVSVLWHW NAPVGLYDTE
ENKWWSGFYT RATDFDIAAT LANPQGANYT LLIRDIDAIA
VQLKRLEAAG VPVLWRPLHE AEGGWFWWGA KGPEPAKQLW
DILYERLTVH HGLDNLIWVW NSILEDWYPG DDTVDILSAD
VYAQGNGPMS TQYNELIALG RDKKMIAAAE VGAAPLPGLL
QAYQANWLWF AVWGDX⁴¹⁶FINN PSWNTVAVLN EIYNSDYVLT
LDEIQGWRS
Where:
the residue at position 140 is a proline or
a leucine (X140 = P or L),
and
the residue at position 416 is an aspartate
or a glycine (X416 = D or G).

SEQ ID NO: 13 differs by at least one amino acid from SEQ ID NO: 11.

In a preferred embodiment, the mutated mannanase of the invention is defined by the sequence SEQ ID NO: 14.

SEQ ID NO: 14
KPCKPRDGPV TYEAEDAILT GTTVDTAQVG YTGRGYVTGF
DEGSDKITFQ ISSATTKLYD LSIRYAAIYG DKRTNVVLNN
GAVSEVFFPA GDSFTSVAAG QVLLNAGQNT IDIVNNWGWY
LIDSITLTPS APRPPHDINL NLNNPNADTN AKKLYSYLRS
VYGNKIISGQ QELHHAEWIR QQTGKTPALV AVDLMDYSPS
RVERGTTSHA VEDAIAHHNA GGIVSVLWHW NAPVGLYDTE
ENKWWSGFYT RATDFDIAAT LANPQGANYT LLIRDIDAIA
VQLKRLEAAG VPVLWRPLHE AEGGWFWWGA KGPEPAKQLW
DILYERLTVH HGLDNLIWVW NSILEDWYPG DDTVDILSAD
VYAQGNGPMS TQYNELIALG RDKKMIAAAE VGAAPLPGLL
QAYQANWLWF AVWGDGFINN PSWNTVAVLN EIYNSDYVLT
LDEIQGWRS

The inventors have shown that the catalytic effectiveness of a polypeptide defined by SEQ ID NO: 14 (P140L/D416G mutant) is increased by a factor of 1.3 (30% increase) compared to that of native mannanase Man5A (SEQ ID NO: 11) on the hydrolysis reaction of galactomannan.

Another object of the invention concerns a polynucleotide encoding a polypeptide of the invention.

According to the invention, said polynucleotide is a DNA or RNA molecule.

In a preferred embodiment of the invention, said polynucleotide encodes a mutated mannanase of the invention defined by a sequence chosen from sequences SEQ ID NO: 3-10 and 13-14.

In a preferred embodiment of the invention, said polynucleotide encodes a sequence chosen from sequences SEQ ID NO: 15-19.

SEQ ID NO: 15
CTCCCCCAAGCACAAGGTGGAGGAGCAGCCGCCTCAGCCAAAGTCAGCGG
CACCCGCTTCGTGATCGACGGCAAAACCGGCTACTTTGCAGGAACAAACT
CCTACTGGATTGGCTTCCTGACCAACAACAGAGATGTCGACACAACCTTG
GACCACATCGCCTCCTCGGGCCTCAAAATCCTCCGCGTCTGGGGCTTCAA
CGACGTGAACAACCAACCATCCGGTAACACCGTCTGGTTCCAACGCCTCG
CCTCCTCAGGCTCCCAAATCAACACCGGCCCCAACGGCCTCCAACGCCTC
GACTACCTCGTCAGATCAGCCGAAACCCGCGGCATCAAGCTCATCATCGC
GCTGGTCAACTACTGGGATGACTTTGGCGGCATGAAAGCCTACGTCAACG
CCTTTGGAGGCACAAAAGAATCCTGGTACACCAACGCCCGCGCTCAGGAG
CAGTACAAGCGTTACATCCAGGCTGTCGTCTCGCGATATGTCAACAGCCC
CGCAATCTTTGCGTGGGAACTTGCCAACGAGCCCAGGTGCAAGGGGTGCA
ACACGAATGTTATTTTCAACTGGGCGACGCAGATTTCAGATTATATCCGG
AGCTTGGATAAGGATCATTTGATCACCCTTGGGGATGAGGGGTTTGGGTT
GCCGGGGCAGACGACGTATCCGTATCAGTATGGGGAGGGGACCGACTTCG
TCAAGAATCTGCAGATTAAGAATCTGGACTTTGGGACGTTTCATATGTAT
CCTGGTCATTGGGGGGTGCCGACGAGTTTTGGTCCAGGGTGGATTAAGGA
TCATGCGGCGGCTTGCAGGGCGGCGGGGAAGCCGTGTTTGTTGGAGGAGT
ATGGGTATGAGAGTGATAGGTGTAATGTGCAGAAGGGCTGGCAGCAGGCG
TCGAGGGAGCTGAGCAGGGATGGGATGAGTAGTGATTTGTTTTGGCAATG
GGGCGATCAGTTGAGTACTGGGCAGACACATAATGATGGGTTCACGATTT
ATTATGGTTCTTCGTTGGCTACTTGCTTGGTTACTGACCATGTGAGGGCT
ATCAATGCTCTCCCGGCG
SEQ ID NO: 16
CTCCCCCAAGCACAAGGTGGAGGAGCAGCCGCCTCAGCCAAAGTCAGCGG
CACCCGCTTCGTGATCGACGGCAAAACCGGCTACTTTGCAGGAACAAACT
CCTACTGGATTGGCTTCCTGACCAACAACAGAGATGTCGACACAACCTTG
GACCACATCGCCTCCTCGGGCCTCAAAATCCTCCGCGTCTGGGGCTTCAA
CGACGTGAACAACCAACCATCCGGTAACACCGTCTGGTTCCAACGCCTCG
CCTCCTCAGGCTCCCAAATCAACACCGGCCCCAACGGCCTCCAACGCCTC
GACTACCTCGTCAGATCAGCCGAAACCCGCGGCATCAAGCTCATCATCGC
GCTGGTCAACTACTGGGATGACTTTGGCGGCATGAAAGCCTACGTCAACG
CCTTTGGAGGCACAAGAGAATCCTGGTACACCAACGCCCGCGCTCAGGAG
CAGTACAAGCGTTACATCCAGGCTGTCGTCTCGCGACATGTCAACAGCCC
CGCAATCTTTGCGTGGGAACTTGCCAACGAGCCCAGGTGCAAGGGGTGCA
ACACGAATGTTATTTTCAACTGGGCGACGCAGATTTCAGATTATATCCGG
AGCTTGGATAAGGATCATTTGATCACCCTTGGGGATGAGGGGTTTGGGTT
GCCGGGGCAGACGACGTATCCGTATCAGTATGGGGAGGGGACCGACTTCG
TCAAGAATCTGCAGATTAAGAATCTGGACTTTGGGACGTTTCATATGTAT
CCTGGTCATTGGGGGGTGCCGACGAGTTTTGGTCCAGGGTGGATTAAGGA
TCATGCGGCGGCTTGCAGGGCGGCGGGGAAGCCGTGTTTGTTGGAGGAGT
ATGGGTATGAGAGTGATAGGTGTAATGTGCAGAAGGGCTGGCAGCAGGCG
TCGAGGGAGCTGAGCAGGGATGGGATGAGTGGTGATTTGTTTTGGCAATG
GGGCGATCAGTTGAGTACTGGGCAGACACATAATGATGGGTTCACGATTT
ATTATGGTTCTTCGTTGGCTACTTGCTTGGTTACTGACCATGTGAGGGCT
ATCAATGCTCTCCCGGCG
SEQ ID NO: 17
CTCCCCCAAGCACAAGGTGGAGGAGCAGCCGCCTCAGCCAAAGTCAGCGG
CACCCGCTTCGTGATCGACGGCAAAACCGGCTACTTTGCAGGAACAAACT
CCTACTGGATTGGCTTCCTGACCAACAACAGAGATGTCGACACAACCTTG
GACCACATCGCCTCCTCGGGCCTCAAAATCCTCCGCGTCTGGGGCTTCAA
CGACGTGAACAACCAACCATCCGGTAACACCGTCTGGTTCCAACGCCTCG
CCTCCTCAGGCTCCCAAATCAACACCGGCCCCAACGGCCTCCAACGCCTC
GACTACCTCGTCAGATCAGCCGAAACCCGCGGCATCAAGCTCATCATCGC
GCTGGTCAACTACTGGGATGACTTTGGCGGCATGAAAGCCTACGTCAACG
CCTTTGGAGGCACAAAAGAATCCTGGTACACCAACGCCCGCGCTCAGGAG
CAGTACAAGCGTTACATCCAGGCTGTCGTCTCGCGATATGTCAACAGCCC
CGCAATCTTTGCGTGGGAACTTGCCAACGAGCCCAGGTGCAAGGGGTGCA
ACACGAATGTTATTTTCAACTGGGCGACGCAGATTTCAGATTATATCCGG
AGCTTGGATAAGGATCATTTGATCACCCTTGGGGATGAGGGGTTTGGGTT
GCCGGGGCAGACGACGTATCCGTATCAGTATGGGGAGGGGACCGACTTCG
TCAAGAATCTGCAGATTAAGAATCTGGACTTTGGGACGTTTCATATGTAT
CCTGGTCATTGGGGGTTGCCGACGAGTTTTGGTCCAGGGTGGATTAAGGA
TCATGCGGCGGCTTGCAGGGCGGCGGTGAAGCCGTGTTTGTTGGAGGAGT
ATGGGTATGAGAGTGATAGGTGTAATGTGCAGAAGGGCTGGCAGCAGGCG
TCGAGGGAGCTGAGCAGGGATGGGATGAGTGGTGATTTGTTTTGGCACTG
GGGCGATCAGTTGAGTACTGGGCAGACACATAATGATGGGTTCACGATTT
ATTATGGTTCTTCGTTGGCTACTTGCTTGGTTACTGACCATGTGAGGGCT
ATCAATGCTCTCCCGGCG
SEQ ID NO: 18
CTCCCCCAAGCACAAGGTGGAGGAGCAGCCGCCTCAGCCAAAGTCAGCGG
CACCCGCTTCGTGATCGACGGCAAAACCGGCTACTTTGCAGGAACAAACT
CCTACAGGATTGGCTTCCTGACCAACAACAGAGATGTCGACACAACCTTG
GACCACATCGCCTCCTCGGGCCTCAAAATCCTCCGCGTCTGGGGCTTCAA
CGACGTGAACAACCAACCATCCGGTAACACCGTCTGGTTCCAACGCCTCG
CCTCCTCAGGCTCCCAAATCAACACCGGCCCCAACGGCCTCCAACGCCTC
GACTACCTCGTCAGATCAGCCGAAACCCGCGGCATCAAGCTCATCATCGC
GCTGGTCAACTACTGGGATGACTTTGGCGGCATGAAAGCCTACGTCAACG
CCTTTGGAGGCACAAAAGAATCCTGGTACACCAACGCCCGCGCTCAGGAG
CAGTACAAGCGTTACATCCAGGCTGTCGTCTCGCGATATGTCAACAGCCC
CGCAATCTTTGCGTGGGAACTTGCCAACGAGCCCAGGTGCAAGGGGTGCA
ACACGAATGTTATTTTCAACTGGGCGACGCAGATCTCAGATTATATCCGG
AGCTTGGATAAGGATCATTTGATCACCCTTGGGGATGAGGGGTTTGGGTT
GCCGGGGCAGACGACGTATCCGTATCAGTATGGGGAGGGGACCGACTTCG
TCAAGAATCTGCAGATTAAGAATCTGGACTTTGGGACGTTTCATATGTAT
CCTGGTCATTGGGGGGCGCCGACGAGTTTTGGTCCAGGGTGGATTAAGGA
TCATGCGGCGGCTTGCAGGGCGGCGGGGAAGCCGTGTTTGTTGGAGGAGT
ATGGGTATGAGAGTGATAGGTGTAATGTGCAGAAGGGCTGGCAGCAGGCG
TCGAGGGAGCTGAGCAGGGATGGGATGAGTGGTGATTTGTTTTGGCAATG
GGGCGATCAGTTGAGTACTGGGCAGACACATAATGATGGGTTCACGATTT
ATTATGGTTCTTCGTTGGCTACTTGCTTGGTTACTGACCATGTGAGGGCT
ATCAATGCTCTCCCGGCG
SEQ ID NO: 19
AAGCCTTGCAAGCCTCGTGATGGCCCCGTGACCTACGAGGCCGAAGATGC
CATCCTCACCGGCACCACCGTCGACACTGCTCAGGTAGGCTATACCGGCC
GTGGCTACGTCACCGGCTTCGACGAGGGCTCCGACAAGATCACCTTCCAG
ATCAGCTCCGCCACCACCAAGCTCTACGACCTCTCCATCCGGTACGCCGC
CATCTACGGTGACAAGCGAACCAACGTCGTCCTCAACAACGGTGCCGTCA
GCGAGGTCTTCTTCCCCGCCGGTGACTCTTTTACTTCTGTCGCCGCCGGC
CAGGTCCTCCTCAACGCTGGACAAAACACCATCGACATCGTCAACAACTG
GGGATGGTACCTCATCGACTCCATCACCCTCACCCCCTCCGCCCCTCGCC
CCCCCCACGACATCAACCCCAACCTCAATAACCCCAACGCCGACACCAAC
GCCAAGAAGCTCTACTCCTACCTCCGCTCTGTCTACGGCAACAAGATCAT
CTCTGGCCAGCAGGAGCTCCACCACGCCGAGTGGATCAGACAGCAAACCG
GCAAGACTCCCGCGCTGGTGGCTGTCGATCTGATGGATTACTCCCCCTCC
CGCGTCGAGCGTGGCACCACCAGCCATGCCGTCGAGGACGCCATCGCCCA
CCACAACGCAGGCGGTATCGTTTCTGTCCTCTGGCACTGGAACGCTCCCG
TCGGTCTGTATGACACCGAAGAGAACAAGTGGTGGTCCGGCTTCTACACT
CGGGCTACCGACTTTGACATTGCCGCCACGTTGGCCAACCCCCAGGGTGC
GAACTACACTCTTCTCATCAGGGACATTGACGCGATTGCTGTCCAGCTCA
AGAGGCTGGAGGCTGCTGGTGTTCCGGTCTTGTGGAGACCTCTTCACGAG
GCGGAGGGTGGTTGGTTCTGGTGGGGAGCCAAGGGGCCAGAGCCGGCGAA
GCAGCTTTGGGATATCTTGTATGAGCGTCTGACGGTGCACCATGGTTTGG
ATAATTTGATTTGGGTGTGGAATTCGATTTTGGAGGATTGGTATCCGGGT
GATGATACGGTTGATATCTTGTCGGCCGATGTGTATGCGCAGGGTAATGG
GCCCATGTCGACTCAGTACAATGAGTTGATCGCCCTCGGCAGGGACAAGA
AGATGATTGCTGCTGCAGAGGTTGGCGCTGCCCCTTTGCCCGGCTTGTTG
CAGGCTTACCAGGCCAACTGGCTGTGGTTTGCTGTCTGGGGTGATGACTT
TATCAGCAACCCCAGCTGGAACACGGTTGCTGTTCTCAACGAGATCTACA
ACAGCGACTATGTGTTGACGCTGGATGAGATTCAGGGGTGGAGGAGT

A polynucleotide of the invention is preferably an isolated and/or purified sequence.

The invention further relates to a vector comprising a polynucleotide of the invention.

The term “vector” (or “plasmid” and “expression vector”) refers to a nucleic acid molecule into which it is possible to insert foreign fragments of nucleic acid, then to introduce them, maintain them and express them in a host cell.

A polynucleotide of the invention can be introduced into any suitable vector for its expression, such as a plasmid, a cosmid, an episome, an artificial chromosome, a phage or a viral vector.

The choice of vectors usable in the context of the present invention is vast. They can be cloning and/or expression vectors. In general, they are known to those skilled in the art and many of them are commercially available but it is also possible to construct them or to modify them by genetic engineering techniques. Plasmids such as JMP61, pPICZαA, pPICZαB, pPICZαC . . . can be quoted by way of example.

Preferably, a vector used in the context of the present invention contains a replication origin ensuring the initiation of replication in a producing cell and/or a host cell. It also contains the elements necessary for the expression of a polynucleotide of the invention, such as a promoter and a terminator. Examples of suitable promoter according to the invention include, but are not limited to promoters POX2, AOX (alcohol oxidase).

It may further comprise one or more selection gene(s) to select or identify the transfected cells with said vector (complementation of an auxotrophic mutation, gene encoding resistance to an antibiotic . . . ). It can also comprise additional elements improving its maintenance and/or its stability in a given cell (cer sequence which promotes the monomeric maintenance of a plasmid, integration sequences into the cell genome).

The vector of the invention may optionally be associated with one or more substances improving the transfectional efficiency and/or the stability of the vector. These substances are widely documented in the literature accessible to those skilled in the art. By way of illustration but without limitation, they may be polymers, in particular cationic lipids, liposomes, nuclear proteins or neutral lipids. These substances may be used alone or in combination. One possible combination is a plasmid recombinant vector associated with cationic lipids (DOGS, DC-CHOL, spermine-chol, spermidine-chol etc.) and neutral lipids (DOPE).

The present invention also relates to a host cell comprising a vector or a polynucleotide of the invention.

For the purposes of the present invention, such a cell is formed of any transfectable cell with a polynucleotide or vector of the invention as described above.

The bacterial expression systems can be used in the context of the present invention. Examples of bacterial host cells include bacteria of the genera Escherichia (e.g. Escherichia coli), Pseudomonas (for example Pseudomonas fluorescens or Pseudomonas stutzerei), Proteus (for example Proteus mirabilis), Ralstonia (for example Ralstonia eutropha), Streptomyces, Staphylococcus (for example Streptomyces carnosus), Lactococcus (for example Lactoccocus lactis) or Bacillus (for example Bacillus subtilis, Bacillus megaterium or Bacillus licheniformis), etc.

Yeast cells are also hosts cells which can be suitable in the scope of the invention. Examples of yeast host cells may be used include, but are not limited to, Saccharomyces cerevisiae. Schizosaccharomyces pombe, Klyveromyces lactis, Yarrowia lipolytica, Hansenula polymorpha or Pichia pastoris.

Fungal expression systems are also conceivable within the scope of the present invention, such as Aspergillus Niger, Chrysosporium lucknowense, Aspergillus (for example Aspergillus oryzae, Aspergillus niger, Aspergillus nidulans, etc.), Podospora anserina or Trichoderma reesei.

Other expression systems such as mammalian expression systems can also be used in the context of the invention, such as cell lines NSO, CHO, BHK, transgenic systems of mammalian origin, but also insect cells or viral expression systems such as the M13, T7 or λ bacteriophages or Baculovirus expression systems.

Preferably, the host cell of the invention is selected from Yarrowia lipolytica and Pichia pastoris.

The polynucleotide contained in the vector and/or the host cell of the invention may optionally be combined with a sequence encoding a signal peptide (also called signal sequence), allowing the secretion of the mutated mannanases of the invention in the extracellular space as well as simplified detection and purification thereof in the culture supernatant of the host cells.

Promoter sequences and signal used in the context of the present invention may be modified and improved by optimisation techniques of sequences well-known to the skilled person.

Another object of the invention relates to a method for producing a polypeptide of the invention, said method comprising the steps of:
(i) production and amplification of a nucleic acid fragment comprising a polynucleotide of the invention,
(ii) inserting said nucleic acid fragment obtained in step (i) in an expression vector comprising a promoter, to enable expression of said nucleic acid fragment under the control of said promoter,
(iii) introducing the expression vector obtained in step (ii) in a host cell,
(iv) culturing the host cell obtained in step (iii),
(v) recovering the polypeptide of the invention.

It is easy for a skilled person to produce a nucleic acid fragment comprising a polynucleotide of the invention.

In a preferred embodiment of the invention, the nucleic acid fragment of step (i) comprises a polynucleotide associated with a signal sequence. Preferably, the signal sequence is fused to the polynucleotide of the invention upstream thereof.

Examples of signal sequences include, but are not limited to the sequence of preprolip2 secretory peptide (Bordes et al., 2007, J Microbiol Meth., 70, 493), the secretion sequence of the pre-pro-factor α of S. cerevisiae (Kjeldsen, 2000 (Appl Microbiol Biotechnol., 54 (3):277-86) or the signal sequence of the native proteins.

The amplification methods of a nucleic acid fragment are well-known to the skilled person, and include the polymerase chain reaction or PCR.

An example of the primer pairs used for amplification of a polypeptide of the invention derived from the mannanase Man5A is provided by SEQ ID NOs: 20-21; an example of the primer pairs used for amplification of a polypeptide of the invention derived from the mannanase Man26A provided by the sequences SEQ ID NO: 22-23.

SEQ ID NO: 20
TTTGAATTCCTCCCCCAAGCACAAGGTG
SEQ ID NO: 21
TTTTCTAGACCCGCCGGGAGAGCATTGATAG
SEQ ID NO: 22
TTTATCGATAAAGCCTTGTAAGCCTCGTG
SEQ ID NO: 23
TTTTCTAGACCACTCCTCCACCCCTGAATCTC

The techniques for inserting a nucleic acid fragment into an expression vector are well-known to those skilled in the art.

Said sequence is inserted into the vector so as to be operably linked to the promoter present in the vector, thereby permitting expression of said nucleic acid sequence under the control of said promoter.

Generally, the term “operably linked” means that the promoter is positioned relative to the inserted nucleic acid fragment so that transcription can begin. This means that the promoter is positioned upstream of said nucleic acid fragment, at a distance allowing the expression of the latter.

In a preferred embodiment of the invention, the expression vector comprises a selection gene.

Examples of selection genes include, but are not limited to the zeocin resistance gene and the histidine-based auxotrophy gene.

Preferably, the expression vector used in step (ii) is JMP61, pPICZαA or pPICZαC.

Step (iii) introducing the vector into the host cell is accomplished by well-known processing techniques of the art, such as electrolocation, transfection, lipofection, chemical transfection, transformation by lithium acetate, biolistic transformation, PEG transformation, protoplast fusion, liposome transformation, transformation by Agrobacterium tumefaciens, viral or adenoviral transformation or still transduction.

Preferably, the host cell of step (iii) is Pichia pastoris or Yarrowia lipolytica.

Where the host cell is Pichia pastoris, the vector of step (ii) is preferably pPICZαA or pPICZαC. Where the host cell is Yarrowia lipolytica, the vector of step (ii) is preferably JMP61.

In case the vector comprises a selection gene, the production method of the invention may comprise an additional step (iv′) for selecting cells that have incorporated the expression vector in step (iii) to increase the production yield of a polypeptide of the invention. This type of selection is carried out by techniques well-known to the man of the art.

Recovering the polypeptide of the invention is made from the culture medium of step (iv) and produced by techniques well-known to those skilled in the art.

If the nucleic acid fragment of step (i) comprises the polynucleotide of the invention associated with a signal sequence, the recovery can be done directly in the secretome of the host cells present in the culture medium obtained in step (iv).

If the nucleic acid fragment of step (i) does not comprise a signal sequence, it will be necessary to lyse the cells and to recover the polypeptide of the invention for example in the supernatant of the culture medium after centrifugation thereof.

The invention further relates to a composition comprising at least one polypeptide, one polynucleotide, one vector or one host cell of the invention.

According to the invention, the composition may comprise one or more polypeptide(s) of the invention (or a polynucleotide, a vector or a host cell of the invention).

The composition may also include, optionally, one or more native mannanase(s) (or at least one or more polynucleotide(s), vector(s) or associated host cell(s)) of Podospora anserina, such as, for example, the native mannanases Man5A and Man26A.

Said composition may also include, optionally, one or more native or mutated mannanase(s) (or at least one or more polynucleotide(s), vector(s) or associated host cell(s)) of any species.

In a particular embodiment of the invention, said composition optionally comprises one or more other hydrolase(s) (or associated polynucleotides, vectors or host cells), whereas the said other hydrolase(s) being useful for the degradation of the lignocellulosic biomass, such as endoglucanases, exoglucanases, mono-oxygenase polysaccharides, β-glucosidases, dehydrogenases cellobiose, xylanases, arabinofuranosidases, galactosidases, arabinanases, esterase carbohydrates, glucuronidases, glucuronoyl methyl esterase, acetyl esterases, pectinases.

In a more particular embodiment of the invention, said composition comprises an enzyme cocktail of Trichoderma reesei cellulases.

In a preferred embodiment, the enzyme cocktail of Trichoderma reesei cellulases is the secretome of said Trichoderma reesei fungus.

The term “secretome” refers to all proteins released by one cell, tissue or organism. The methods to recover the secretome of a cell, including the methods of obtaining a cocktail of Trichoderma reesei cellulases are well-known to those skilled in the art. Such cocktails of Trichoderma reesei cellulases are also commercially available, such as the following enzyme cocktails: GC220 (GENENCOR), MULTIFECT GC (GENENCOR), Accellerase (Danisco), Cellic C-Tec (NOVOZYME) or still CELLUCLAST 1.5L (NOVOZYME).

In another particular embodiment of the invention, said composition further comprises an enzyme cocktail of Trichoderma reesei cellulase and an enzyme cocktail of Pycnoporus cinnabarinus, comprising a dehydrogenase cellobiose (CDH) or a dehydrogenase cellobiose of Pycnoporus cinnabarinus (or an associated polynucleotide, vector or host cell).

In a preferred embodiment, said composition comprises an enzyme cocktail of Trichoderma reesei cellulases and at least one polypeptide of the invention at a concentration of at most 10 mg polypeptide per gram of material to be hydrolysed, preferably 10 mcg polypeptide per gram of material to be hydrolysed.

The present patent application is also intended to cover the various possible uses of a polypeptide, a polynucleotide, a vector, a host cell and/or a composition of the invention.

Mannanases are enzymes currently used in a variety of industrial fields such as the paper and cellulose industry, the agriculture and food industry, coffee extraction, oil drilling or the detergent industry.

Thus, an object of the invention relates to the use of a polypeptide, a polynucleotide, a vector, a host cell and/or a composition of the invention for the degradation of compounds comprising mannans.

A mannan is a polysaccharide composed of mannose monomers. The term “mannan” is here used in the broad sense, and encompasses complex sugars and derivatives comprising mannose polymers. In particular, the term includes simple mannans (polymers consisting solely of mannose), galactomannans, glucomannan and galactoglucomannans.

Mannans are found in many plant compounds, such as fruit and certain algae. They are also abundant in certain seeds and nuts (“ivory nut”, locust bean gum, tara gum, guar gum, fenurec gum). They are especially an important component of the biomass, particularly lignocellulosic biomass such as softwoods (gymnosperms), resinous woods (pine, spruce) and in significant amounts in hardwoods.

In the field of energy, especially bioenergy, the term biomass refers to all organic materials of plant (including algae), animal or fungal origin can become a source of energy, for example by combustion after methanisation (biogas) or after new chemical transformations (agrofuel). One of its main components is lignocellulosic biomass.

The term “lignocellulosic biomass” refers to a material derived from plants or other organisms in which the carbohydrate content is substantially lignocellulose consisting of cellulose, hemicellulose and lignin (equivalent to at least 5%). It consists for example of wood and green waste, straw, straw briquettes, sugar cane bagasse, or fodder. In particular lignocellulosic biomass include processed materials, such as paper having more than 5% lignin, but also natural raw materials, such as agricultural waste. A mixture of water and/or other agents and solvents comprising lignocellulosic biomass as a main solid component can also be considered as lignocellulosic biomass as such.

In a preferred embodiment of the invention, lignocellulosic biomass is selected from the group consisting of herbaceous agricultural residues, forestry residues, municipal solid waste, paper type waste, pulp and paper mill residues, or any combination thereof.

In another preferred embodiment, the lignocellulosic biomass according to the invention is selected from a group consisting of corn cobs and stalks, straw e.g. from rice, wheat, rye, oats, barley, lavandin, bagasse, miscanthus, herbs, bamboo, water hyacinth, wood consisting of hardwood such as eucalyptus, poplar coppice, wood consisting of softwood for example acacia, soft wood pulp . . . .

In a particular embodiment, the invention relates to the use of a polypeptide, a polynucleotide, a vector, a host cell and/or a composition of the invention for the degradation of lignocellulosic biomass.

In a more particular embodiment, the invention relates to the use of a polypeptide, a polynucleotide, a vector, a host cell and/or a composition of the invention for the pretreatment lignocellulosic biomass to be degraded.

The term “pretreatment” means a manipulation of lignocellulosic biomass which makes its components cellulose more accessible to enzymes converting carbohydrate polymers into fermentable sugars.

In a preferred embodiment, the invention relates to the use of a composition comprising a polypeptide of the invention (or a polynucleotide, a vector or a host cell of the invention) and an enzyme cocktail of Trichoderma reesei cellulases for the degradation of the lignocellulosic biomass and/or for the pretreatment of lignocellulosic biomass to be degraded.

In an even more preferred embodiment, the composition may further comprise other native or mutated mannanases, of Podospora anserina or other species, and other useful enzymes to degradation of lignocellulosic biomass.

In a particular embodiment, the invention relates to the use of a polypeptide, a polynucleotide, a vector, a host cell and/or a composition of the invention for the production of biofuels.

Indeed, the components of lignocellulosic biomass are suitable substrates for the production of biofuels. The polypeptides of the invention are used for converting lignocellulosic biomass, the products thus obtained can be used as biofuels (for example, bioethanol, biobutanol) or as molecular components of such fuels (for example, 3-hydroxy propionic acid, aspartic acid, xylitol and gluconic acid).

In a particular embodiment, the invention relates to the use of a polypeptide, a polynucleotide, a vector, a host cell and/or a composition of the invention for the stimulation of oil and gas wells by hydraulic fracturing.

In another particular embodiment, the invention relates to the use of a polypeptide, a polynucleotide, a vector, a host cell and/or a composition of the invention for the production of mannose or mannan-oligosaccharides from plant compounds containing mannans.

Examples of such compounds include, but are not limited to, oil palm kernel, coconut, copra, konjac, locust bean gum, guar gum, soybean . . . .

Mannose is indeed a relatively rare resource with beneficial properties and used in food, pharmaceuticals, cosmetics, textiles and the manufacture of polymers. It may especially be used as a raw material for the production of mannitol.

In a more particular embodiment, the invention relates to the use of a polypeptide, a polynucleotide, a vector, a host cell and/or a composition of the invention for the production of mannitol.

Many uses are possible in the food and agriculture industry.

In a particular embodiment, the invention relates to the use of a polypeptide, a polynucleotide, a vector, a host cell and/or a composition of the invention as a dietary supplement.

Indeed, mannanases promote the degradation of food components containing mannans, thus releasing oligomannoses known to have beneficial properties for human and animal health and facilitate the digestion by breaking normally hardly degradable polymers by polymers; they are particularly useful to the body as prebiotics.

In another particular embodiment, the invention relates to a polypeptide, a polynucleotide, a vector, a host cell and/or a composition of the invention in the treatment of food components.

Indeed, the application of mannanases to pineapples, lemons, oranges, grapefruits before pressing them enables improving the recovery of juice from these pieces of fruit.

Also, mannanases can also be used in connection with the extraction of palm oil: their application to cakes of oil palm kernels, after a first extraction pressure, allows improving the yield but also obtaining better quality oil palm kernel cake (because they contain less fibre galactomannans, anti-nutrient components in animal feed).

In another particular embodiment, the invention relates to the use of a polypeptide, a polynucleotide, a vector, a host cell and/or a composition of the invention for the extraction of palm oil.

In another particular embodiment, the invention relates to the use of a polypeptide, a polynucleotide, a vector, a host cell and/or a composition of the invention for the extraction of coffee.

The use of mannanases in the extraction of coffee enables the hydrolysis of galactomannans present in the liquid coffee extract, thereby enabling to reduce the viscosity of these liquid extracts and to decrease the consumption of enzymes and energy during extraction.

Furthermore, in connection with the extraction of the coffee, the waste may be used for the production of mannose as described above.

In a particular embodiment, the invention relates to the use of a polypeptide, a polynucleotide, a vector, a host cell and/or a composition of the invention as a cooking supplement.

In another particular embodiment, the invention relates to the use of a polypeptide, a polynucleotide, a vector, a host cell and/or a composition of the invention for the bleaching of pulp.

A polypeptide, a polynucleotide, a vector, a host cell and/or a composition of the invention can be used alone or in combination with one or more other native or mutated mannanase(s) (associated polypeptide(s), polynucleotide(s), vector(s), host cell(s) and/or composition(s)), with xylanases, endoglucanases, α-galactosidases, cellobiohydrolases . . . .

In another particular embodiment, the invention relates to the use of a polypeptide, a polynucleotide, a vector, a host cell and/or a composition of the invention for desizing and bleaching textile fibres.

In another particular embodiment, the invention relates to the use of a polypeptide, a polynucleotide, a vector, a host cell and/or a composition of the invention in detergent compositions.

According to the invention, a polypeptide, a polynucleotide, a vector, a host cell and/or a composition of the invention can be used alone or in combination with other native or mutated mannanases, amylases, cellulases, lipases, pectinases, proteases and endoglucanases.

Mannanases also show the properties of interest for the pharmaceutical industry.

In another particular embodiment, the invention relates to the use of a polypeptide, a polynucleotide, a vector, a host cell and/or a composition of the invention for the elimination of biofilms.

For the removal of biofilms, a polypeptide (a polynucleotide, vector, host cell or composition) of the invention can be used alone or in combination with detergents, other native or mutated mannanases, α-galactosidases, pectinases, xylanases, arabinoxylanases, proteases, beta-glucanases, cellulases, galactanases, endoglucanases, xylosidases, cutinases and lipases.

In another particular embodiment, the invention relates to the use of a polypeptide, a polynucleotide, a vector, a host cell and/or a composition of the invention in targeted delivery or time-controlled release systems.

Such systems are widely used in the pharmaceutical industry for the administration of active ingredients up to a given organ and/or for a defined period. They are produced using systems based on mannopolymer gels which contain and carry the material.

The function of a mannanase in such a system is the controlled release of the material by partial or complete degradation of the gel, due to a specific change in the environment of the gel. e.g., pH and/or temperature, which activates mannanases.

Mannanases also show applications in the alcohol fermentation and/or production processes.

Thus, another object of the invention relates to a method for producing a fermentation product from lignocellulosic biomass, said method comprising the steps of:
(i) liquefaction of lignocellulosic biomass by using a polypeptide, a polynucleotide, a vector, a host cell and/or a composition of the invention, in order to obtain a liquefied product having a dry mass at least 20 mass %,
(ii) saccharification of said liquefied product obtained in step (i) with an enzyme cocktail,
(iii) fermentation of the saccharification product obtained in step (ii) using a fermenting microorganism.

In a particular embodiment of the invention, the enzyme cocktail used in step (ii) is a composition of the invention, comprising in particular (a) a polypeptide, a polynucleotide, a vector and/or a host cell and (b) optionally, a cocktail of Trichoderma reesei cellulases.

Another object of the invention relates to a method for producing gluconic acid, xylonic acid and/or xylobionic acid or increased gluconic acid, xylonic acid and/or xylobionic acid from lignocellulosic biomass, said method comprising the steps of:
(i) liquefaction of lignocellulosic biomass by using a polypeptide, a polynucleotide, a vector, a host cell and/or a composition of the invention, in order to obtain a product liquid having a dry mass at least 20 mass %,
(ii) saccharification of said liquefied product obtained in step (i) with an enzyme cocktail.

In a particular embodiment of the invention, the enzyme cocktail used in step (ii) is a composition of the invention, comprising in particular (a) a polypeptide, a polynucleotide, a vector and/or a host cell and (b) optionally, a cocktail of Trichoderma reesei cellulases.

Thus, another object of the invention relates to a method for increasing the production of sugars a from lignocellulosic biomass, said method comprising the steps of:
(i) liquefaction of lignocellulosic biomass by using a polypeptide, a polynucleotide, a vector, a host cell and/or a composition of the invention, in order to obtain a liquefied product having a dry mass at least 20 mass %,
(ii) saccharification of said liquefied product obtained in step (i) with an enzyme cocktail.

In a particular embodiment of the invention, the enzyme cocktail used in step (ii) is a composition of the invention, comprising in particular (a) a polypeptide, a polynucleotide, a vector and/or a host cell and (b) optionally, a cocktail of Trichoderma reesei cellulases.

### Examples

Other characteristics of the invention appear in the following examples, whereas the latter do not constitute any limitation of the invention.

The inventors have developed mutants of the Man5A and Man26A proteins of Podospora anserina and studied their activity, seeking to increase the effectiveness of these enzymes, in particular to increase the efficiency of the enzyme cocktails used for the degradation of the lignocellulosic biomass.

Production of Native Enzymes

Thus the two genes encoding the Man5A proteins (nucleic acid sequence defined by SEQ ID NO: 2) and Man26A (nucleic acid sequence defined by SEQ ID NO: 12) of Podospora anserina were each amplified with the primers identified by SEQ ID NO: 20-21 and 22-23 respectively, inserted into the expression vector JMP61 associated with a preprolip2 secretion peptide (Bordes et al, 2007, J Microbiol Meth., 70, 493) and placed under the control of the oleic acid-inducible promoter POX2. The yeast cells of Yarrowia lipolytica were transformed with the vectors obtained (JMP61-Man5A and JMP61-Man26A). Positive transformants were selected on plates comprising galactomannan. They were able to produce Man5A and Man26A functional enzymes at a level of 10.4+/−0.2 and 11.2+/−0.6 U·mL⁻¹ in vitro.

The mean activities obtained after repeating the experiments were 1.78+/−1.24 and 0.138+/−0.152 U·mL⁻¹ for culturing Man5A and Man26A respectively.

Production of Mutants

The researchers have then developed mutants and studied their activity. Four mutants were developed for Man5A:
the G311S mutant, defined by the protein sequence SEQ ID NO: 4, and encoded by the sequence SEQ ID NO: 15,
the K139R/Y223H mutant, defined by the protein sequence SEQ ID NO: 6, and encoded by the sequence SEQ ID NO:16,
the V256L/G276V/Q316H mutant, defined by the protein sequence SEQ ID NO: 8, and encoded by the nucleic sequence SEQ ID NO: 17, and
the W36R/I195TN256A mutant, defined by the protein sequence SEQ ID NO: 10, and encoded by the nucleic sequence SEQ ID NO:18.
   A single mutant was developed from Man26A:
the P140L/ID416G mutant, defined by the protein sequence SEQ ID NO: 14, and encoded by the nucleic sequence SEQ ID NO: 19.

Study of the Activity of Yarrowia lipolytica Strains Expressing these Mutants

The strain expressing the mutant P140L/D416G of Man26A showed an increased activity on the hydrolysis reaction of galactomannan of 147% compared to a strain expressing the Man26 of the native Podospora anserina.

On the same reaction, the strains expressing the Man5A mutants showed an increased activity of 46, 9, 20 and 11% respectively for V256L/G276V/Q316H. W36R/1195T/V256A, K139R/Y223H, G311S mutant compared to a strain of Yarrowia lipolvtica expressing the Man5A protein of native Podospora anserina.

The inventors then wanted to assess more accurately the importance of these mutants from an enzymatic viewpoint, in particular for the degradation of lignocellulosic biomass. They therefore investigated the hydrolysis profile of galactomannan for each of them.

Production of Mutants in the Pichia pastoris Expression System

The mutants were produced in the Pichia pastoris expression system, for obtaining higher expression levels than those obtained in the Yarrowia lipolytica expression system.

The genes encoding the Man5A and Man26A native proteins of Podospora anserina and the developed mutants were each amplified with the primers defined by the sequences SEQ ID NO: 20-23 (primers defined by the sequences SEQ ID NO: 20-21 for Man5A and its mutants, primers defined by the sequences SEQ ID NO: 22-23 for Man26A and its mutant), inserted into the pPICZαA expression vector, associated with the secretion sequence of the pre-pro α-factor of Saccharomyces cerevisiae (Kjeldsen, 2000 (Appl Microbiol Biotechnol., 54 (3): 277-86) and the C-terminal sequence (His)₆tag, under the control of the promoter AOX (alcohol oxidase). The expression vector used included a resistance gene to zeocin.

Pichia pastoris cells were transformed with the vectors obtained (pPICZαA-Man5A, pPICZαA-Man26A, pPICZαA-Man5A-G311 S, pPICZαA-Man5A-K139R/Y223H, pPICZαA-Man5A-V256L/G276V/Q316H, pPICZαA-Man5A W36R/I1195TN256A, pPICZαA-Man26A-P140L/D416G). Positive transformants were selected due to their resistance to zeocin.

All mutants were successfully produced in the expression system of Pichia pastoris at about 1 g/L of culture medium and purified thanks to C-terminal sequence (His)₆tag.

Kinetic Characterisation of Mutants

For each mutant, the hydrolysis capacity of galactomannan was assessed.

The kinetic parameters of each native or mutated enzyme, on the hydrolysis reaction of galactomannan was determined using the DNS activity test: 1 μg of each native or mutated enzyme, was mixed with 190 μg galactomannan and incubated at 40° C. for 5 minutes. The reaction was stopped by adding 300 μL DNS and the samples were placed for 10 minutes at 95° C. The optical density OD540 was measured relatively to the mannose standard from 0 to 20 mM. One unit of activity mannanase (endo-β1,4-mannanase) was defined as the amount of protein required to release 1 μmol of sugar monomer per minute.

Kinetic parameters were estimated by weighted non-linear regression analysis, using the GRAFIT program. The constants K_{cat}, K_{M}, and the catalytic efficiency were measured for each native or mutated enzyme.

The results are summarised in Table 1:

TABLE 1
Increase in the catalytic efficiency of each mutant compared to
its native enzyme.

| | Catalytic efficiency | |
|---|---|---|
| | K_{cat}/K_{M} | Increase in catalytic |
| Enzyme tested | (mg^{-l}.ml⁻¹.min⁻¹) | efficiency |
| Native Man26A | 1398 | — |
| P140L/D416G | 1838 | +31% |
| Native Man5A | 145 | — |
| V256L/G276V/Q316H | 191 | +31% |
| W36R/I195T/V256A | 259 | +78% |
| K139R/Y223H | 247 | +70% |
| G311S | 1187 | Multiplied by a factor of 8 |

In view of the apparent K_{M} values, all the mutants have showed an improved apparent affinity for galactomannan.

All the mutants show improved catalytic efficiency of at least 30% compared to the native enzyme. The mutant G311S of Man5A shows for its own part a surprising activity, increased by a factor of 8 compared to the native enzyme.

1. A polypeptide having a sequence derived from a native mannanase of the filamentous ascomycete coprophile fungus Podospora anserina, and being characterised by an increased catalyst efficiency of at least 25% compared to the catalytic efficiency of this native mannanase.

2. A polypeptide according to claim 1, characterised in that it is derived from the Man5A or Man26A mannanase of Podospora anserina respectively defined by the protein sequences SEQ ID NO: 1 and SEQ ID NO: 11.

3. A polypeptide according to claim 2, characterised in that it is defined by a sequence chosen from sequences SEQ ID NO: 3-10 or 13-14 or the sequences SEQ ID NO: 3 10 and 13-14 differ by at least one amino acid of SEQ ID NO:1 and 11.

4. A polynucleotide encoding a polypeptide according to claim 1.

5. A polynucleotide according to claim 4, characterised in that it is defined by a sequence chosen from sequences SEQ ID NO: 15-19.

6. A vector comprising a polynucleotide according to claim 4.

7. A host cell comprising a polynucleotide according to claim 4 or a vector comprising said polynucleotide.

8. A composition comprising a polypeptide according to claim 1, a polynucleotide encoding said polypeptide, a vector comprising said polynucleotide or a host cell comprising said vector or said polynucleotide.

9. A composition according to claim 8, characterised in that it further comprises other hydrolases such as mannanases, endoglucanases, exoglucanases, β-glucosidases or xylanases.

10. A composition according to claim 8, characterised in that it comprises an enzyme cocktail of Trichoderma reesei cellulases.

11. A method for degrading compounds comprising mannans, said method comprising treating said compounds with a polypeptide according to claim 1, a polynucleotide encoding said polypeptide, a vector comprising said polynucleotide, a host cell comprising said vector or said polynucleotide or a composition comprising said polypeptide, said polynucleotide, said vector, or said host cell.

12. The method according to claim 11, wherein the compound comprising mannans is a lignocellulosic biomass.


SEQUENCE LISTING (23 sequences)

SEQ ID NO: 1 - protein, 356 aa, Podospora anserina
LPQAQGGGAAASAKVSGTRFVIDGKTGYFAGTNSYWIGFLTNNRDVDTTLDHIASSGLKILRVWGFNDVNNQPSGNTVWFQRLASSGSQINTGPNGLQRLDYLVRSAETRGIKLIIALVNYWDDFGGMKAYVNAFGGTKESWYTNARAQEQYKRYIQAVVSRYVNSPAIFAWELANEPRCKGCNTNVIFNWATQISDYIRSLDKDHLITLGDEGFGLPGQTTYPYQYGEGTDFVKNLQIKNLDFGTFHMYPGHWGVPTSFGPGWIKDHAAACRAAGKPCLLEEYGYESDRCNVQKGWQQASRELSRDGMSGDLFWQWGDQLSTGQTHNDGFTIYYGSSLATCLVTDHVRAINALPA

SEQ ID NO: 2 - DNA, 1070 bp, Podospora anserina
tcccccaagcacaaggtggaggagcagccgcctcagccaaagtcagcggcacccgcttcgtgatcgacggcaaaaccggctactttgcaggaacaaactcctactggattggcttcctgaccaacaacagagatgtcgacacaaccttggaccacatcgcctcctcgggcctcaaaatcctccgcgtctggggcttcaacgacgtgaacaaccaaccatccggtaacaccgtctggttccaacgcctcgcctcctcaggctcccaaatcaacaccggccccaacggcctccaacgcctcgactacctcgtcagatcagccgaaacccgcggcatcaagctcatcatcgcgctggtcaactactgggatgactttggcggcatgaaagcctacgtcaacgcctttggaggcacaaaagaatcctggtacaccaacgcccgcgctcaggagcagtacaagcgttacatccaggctgtcgtctcgcgatatgtcaacagccccgcaatctttgcgtgggaacttgccaacgagcccaggtgcaaggggtgcaacacgaatgttattttcaactgggcgacgcagatttcagattatatccggagcttggataaggatcatttgatcacccttggggatgaggggtttgggttgccggggcagacgacgtatccgtatcagtatggggaggggaccgacttcgtcaagaatctgcagattaagaatctggactttgggacgtttcatatgtatcctggtcattggggggtgccgacgagttttggtccagggtggattaaggatcatgcggcggcttgcagggcggcggggaagccgtgtttgttggaggagtatgggtatgagagtgataggtgtaatgtgcagaagggctggcagcaggcgtcgagggagctgagcagggatgggatgagtggtgatttgttttggcaatggggcgatcagttgagtactgggcagacacataatgatgggttcacgatttattatggttcttcgttggctacttgcttggttactgaccatgtgagggctatcaatgctctcccggcgtag

SEQ ID NO: 3 - protein, 356 aa, Artificial Sequence
  mutant (Podospora anserina)
LPQAQGGGAAASAKVSGTRFVIDGKTGYFAGTNSYXIGFLTNNRDVDTTLDHIASSGLKILRVWGFNDVNNQPSGNTVWFQRLASSGSQINTGPNGLQRLDYLVRSAETRGIKLIIALVNYWDDFGGMKAYVNAFGGTXESWYTNARAQEQYKRYIQAVVSRYVNSPAIFAWELANEPRCKGCNTNVIFNWATQXSDYIRSLDKDHLITLGDEGFGLPGQTTXPYQYGEGTDFVKNLQIKNLDFGTFHMYPGHWGXPTSFGPGWIKDHAAACRAAXKPCLLEEYGYESDRCNVQKGWQQASRELSRDGMSXDLFWXWGDQLSTGQTHNDGFTIYYGSSLATCLVTDHVRAINALPA

SEQ ID NO: 4 - protein, 356 aa, Artificial Sequence
  mutant (Podospora anserina)
LPQAQGGGAAASAKVSGTRFVIDGKTGYFAGTNSYWIGFLTNNRDVDTTLDHIASSGLKILRVWGFNDVNNQPSGNTVWFQRLASSGSQINTGPNGLQRLDYLVRSAETRGIKLIIALVNYWDDFGGMKAYVNAFGGTKESWYTNARAQEQYKRYIQAVVSRYVNSPAIFAWELANEPRCKGCNTNVIFNWATQISDYIRSLDKDHLITLGDEGFGLPGQTTYPYQYGEGTDFVKNLQIKNLDFGTFHMYPGHWGVPTSFGPGWIKDHAAACRAAGKPCLLEEYGYESDRCNVQKGWQQASRELSRDGMSSDLFWQWGDQLSTGQTHNDGFTIYYGSSLATCLVTDHVRAINALPA

SEQ ID NO: 5 - protein, 356 aa, Artificial Sequence
  mutant (Podospora anserina)
LPQAQGGGAAASAKVSGTRFVIDGKTGYFAGTNSYWIGFLTNNRDVDTTLDHIASSGLKILRVWGFNDVNNQPSGNTVWFQRLASSGSQINTGPNGLQRLDYLVRSAETRGIKLIIALVNYWDDFGGMKAYVNAFGGTXESWYTNARAQEQYKRYIQAVVSRYVNSPAIFAWELANEPRCKGCNTNVIFNWATQISDYIRSLDKDHLITLGDEGFGLPGQTTXPYQYGEGTDFVKNLQIKNLDFGTFHMYPGHWGVPTSFGPGWIKDHAAACRAAGKPCLLEEYGYESDRCNVQKGWQQASRELSRDGMSGDLFWQWGDQLSTGQTHNDGFTIYYGSSLATCLVTDHVRAINALPA

SEQ ID NO: 6 - protein, 356 aa, Artificial Sequence
  mutant (Podospora anserina)
LPQAQGGGAAASAKVSGTRFVIDGKTGYFAGTNSYWIGFLTNNRDVDTTLDHIASSGLKILRVWGFNDVNNQPSGNTVWFQRLASSGSQINTGPNGLQRLDYLVRSAETRGIKLIIALVNYWDDFGGMKAYVNAFGGTRESWYTNARAQEQYKRYIQAVVSRYVNSPAIFAWELANEPRCKGCNTNVIFNWATQISDYIRSLDKDHLITLGDEGFGLPGQTTHPYQYGEGTDFVKNLQIKNLDFGTFHMYPGHWGVPTSFGPGWIKDHAAACRAAGKPCLLEEYGYESDRCNVQKGWQQASRELSRDGMSGDLFWQWGDQLSTGQTHNDGFTIYYGSSLATCLVTDHVRAINALPA

SEQ ID NO: 7 - protein, 356 aa, Artificial Sequence
  mutant (Podospora anserina)
LPQAQGGGAAASAKVSGTRFVIDGKTGYFAGTNSYWIGFLTNNRDVDTTLDHIASSGLKILRVWGFNDVNNQPSGNTVWFQRLASSGSQINTGPNGLQRLDYLVRSAETRGIKLIIALVNYWDDFGGMKAYVNAFGGTKESWYTNARAQEQYKRYIQAVVSRYVNSPAIFAWELANEPRCKGCNTNVIFNWATQISDYIRSLDKDHLITLGDEGFGLPGQTTYPYQYGEGTDFVKNLQIKNLDFGTFHMYPGHWGXPTSFGPGWIKDHAAACRAAXKPCLLEEYGYESDRCNVQKGWQQASRELSRDGMSGDLFWXWGDQLSTGQTHNDGFTIYYGSSLATCLVTDHVRAINALPA

SEQ ID NO: 8 - protein, 356 aa, Artificial Sequence
  mutant (Podospora anserina)
LPQAQGGGAAASAKVSGTRFVIDGKTGYFAGTNSYWIGFLTNNRDVDTTLDHIASSGLKILRVWGFNDVNNQPSGNTVWFQRLASSGSQINTGPNGLQRLDYLVRSAETRGIKLIIALVNYWDDFGGMKAYVNAFGGTKESWYTNARAQEQYKRYIQAVVSRYVNSPAIFAWELANEPRCKGCNTNVIFNWATQISDYIRSLDKDHLITLGDEGFGLPGQTTYPYQYGEGTDFVKNLQIKNLDFGTFHMYPGHWGLPTSFGPGWIKDHAAACRAAVKPCLLEEYGYESDRCNVQKGWQQASRELSRDGMSGDLFWHWGDQLSTGQTHNDGFTIYYGSSLATCLVTDHVRAINALPA

SEQ ID NO: 9 - protein, 356 aa, Artificial Sequence
  mutant (Podospora anserina)
LPQAQGGGAAASAKVSGTRFVIDGKTGYFAGTNSYXIGFLTNNRDVDTTLDHIASSGLKILRVWGFNDVNNQPSGNTVWFQRLASSGSQINTGPNGLQRLDYLVRSAETRGIKLIIALVNYWDDFGGMKAYVNAFGGTKESWYTNARAQEQYKRYIQAVVSRYVNSPAIFAWELANEPRCKGCNTNVIFNWATQXSDYIRSLDKDHLITLGDEGFGLPGQTTYPYQYGEGTDFVKNLQIKNLDFGTFHMYPGHWGXPTSFGPGWIKDHAAACRAAGKPCLLEEYGYESDRCNVQKGWQQASRELSRDGMSGDLFWQWGDQLSTGQTHNDGFTIYYGSSLATCLVTDHVRAINALPA

SEQ ID NO: 10 - protein, 356 aa, Artificial Sequence
  mutant (Podospora anserina)
LPQAQGGGAAASAKVSGTRFVIDGKTGYFAGTNSYRIGFLTNNRDVDTTLDHIASSGLKILRVWGFNDVNNQPSGNTVWFQRLASSGSQINTGPNGLQRLDYLVRSAETRGIKLIIALVNYWDDFGGMKAYVNAFGGTKESWYTNARAQEQYKRYIQAVVSRYVNSPAIFAWELANEPRCKGCNTNVIFNWATQTSDYIRSLDKDHLITLGDEGFGLPGQTTYPYQYGEGTDFVKNLQIKNLDFGTFHMYPGHWGAPTSFGPGWIKDHAAACRAAGKPCLLEEYGYESDRCNVQKGWQQASRELSRDGMSGDLFWQWGDQLSTGQTHNDGFTIYYGSSLATCLVTDHVRAINALPA

SEQ ID NO: 11 - protein, 449 aa, Podospora anserina
KPCKPRDGPVTYEAEDAILTGTTVDTAQVGYTGRGYVTGFDEGSDKITFQISSATTKLYDLSIRYAAIYGDKRTNVVLNNGAVSEVFFPAGDSFTSVAAGQVLLNAGQNTIDIVNNWGWYLIDSITLTPSAPRPPHDINPNLNNPNADTNAKKLYSYLRSVYGNKIISGQQELHHAEWIRQQTGKTPALVAVDLMDYSPSRVERGTTSHAVEDAIAHHNAGGIVSVLWHWNAPVGLYDTEENKWWSGFYTRATDFDIAATLANPQGANYTLLIRDIDAIAVQLKRLEAAGVPVLWRPLHEAEGGWFWWGAKGPEPAKQLWDILYERLTVHHGLDNLIWVWNSILEDWYPGDDTVDILSADVYAQGNGPMSTQYNELIALGRDKKMIAAAEVGAAPLPGLLQAYQANWLWFAVWGDDFINNPSWNTVAVLNEIYNSDYVLTLDEIQGWRS

SEQ ID NO: 12 - DNA, 1350 bp, Podospora anserina
aagccttgtaagcctcgtgatggccccgtgacctacgaggccgaagatgccatcctcaccggcaccaccgtcgacactgctcaggtaggctataccggccgtggctacgtcaccggcttcgacgagggctccgacaagatcaccttccagatcagctccgccaccaccaagctctacgacctctccatccggtacgccgccatctacggtgacaagcgaaccaacgtcgtcctcaacaacggtgccgtcagcgaggtcttcttccccgccggtgactcttttacttctgtcgccgccggccaggtcctcctcaacgctggacaaaacaccatcgacatcgtcaacaactggggatggtacctcatcgactccatcaccctcaccccctccgcccctcgccccccccacgacatcaaccccaacctcaacaaccccaacgccgacaccaacgccaagaagctctactcctacctccgctctgtctacggcaacaagatcatctctggccagcaggagctccaccacgccgagtggatcagacagcaaaccggcaagactcccgcgctggtggctgtcgatctgatggattactccccctcccgcgtcgagcgtggcaccaccagccatgccgtcgaggacgccatcgcccaccacaacgcaggcggtatcgtttctgtcctctggcactggaacgctcccgtcggtctgtatgacaccgaagagaacaagtggtggtccggcttctacactcgggctaccgactttgacattgccgccacgttggccaacccccagggtgcgaactacactcttctcatcagggacattgacgcgattgctgtccagctcaagaggctggaggctgctggtgttccggtcttgtggagacctcttcacgaggcggagggtggttggttctggtggggagccaaggggccagagccggcgaagcagctttgggatatcttgtatgagcgtctgacggtgcaccatggtttggataatttgatttgggtgtggaattcgattttggaggattggtatccgggtgatgatacggttgatatcttgtcggccgatgtgtatgcgcagggtaatgggcccatgtcgactcagtacaatgagttgatcgccctcggcagggacaagaagatgattgctgctgcagaggttggcgctgcccctttgcccggcttgttgcaggcttaccaggccaactggctgtggtttgctgtctggggtgatgactttatcaacaaccccagctggaacacggttgctgttctcaacgagatctacaacagcgactatgtgttgacgctggatgagattcaggggtggaggagttag

SEQ ID NO: 13 - protein, 449 aa, Artificial Sequence
  mutant (Podospora anserina)
KPCKPRDGPVTYEAEDAILTGTTVDTAQVGYTGRGYVTGFDEGSDKITFQISSATTKLYDLSIRYAAIYGDKRTNVVLNNGAVSEVFFPAGDSFTSVAAGQVLLNAGQNTIDIVNNWGWYLIDSITLTPSAPRPPHDINXNLNNPNADTNAKKLYSYLRSVYGNKIISGQQELHHAEWIRQQTGKTPALVAVDLMDYSPSRVERGTTSHAVEDAIAHHNAGGIVSVLWHWNAPVGLYDTEENKWWSGFYTRATDFDIAATLANPQGANYTLLIRDIDAIAVQLKRLEAAGVPVLWRPLHEAEGGWFWWGAKGPEPAKQLWDILYERLTVHHGLDNLIWVWNSILEDWYPGDDTVDILSADVYAQGNGPMSTQYNELIALGRDKKMIAAAEVGAAPLPGLLQAYQANWLWFAVWGDXFINNPSWNTVAVLNEIYNSDYVLTLDEIQGWRS

SEQ ID NO: 14 - protein, 449 aa, Artificial Sequence
  mutant (Podospora anserina)
KPCKPRDGPVTYEAEDAILTGTTVDTAQVGYTGRGYVTGFDEGSDKITFQISSATTKLYDLSIRYAAIYGDKRTNVVLNNGAVSEVFFPAGDSFTSVAAGQVLLNAGQNTIDIVNNWGWYLIDSITLTPSAPRPPHDINLNLNNPNADTNAKKLYSYLRSVYGNKIISGQQELHHAEWIRQQTGKTPALVAVDLMDYSPSRVERGTTSHAVEDAIAHHNAGGIVSVLWHWNAPVGLYDTEENKWWSGFYTRATDFDIAATLANPQGANYTLLIRDIDAIAVQLKRLEAAGVPVLWRPLHEAEGGWFWWGAKGPEPAKQLWDILYERLTVHHGLDNLIWVWNSILEDWYPGDDTVDILSADVYAQGNGPMSTQYNELIALGRDKKMIAAAEVGAAPLPGLLQAYQANWLWFAVWGDGFINNPSWNTVAVLNEIYNSDYVLTLDEIQGWRS

SEQ ID NO: 15 - DNA, 1068 bp, Artificial Sequence
  mutant (Podospora anserina)
ctcccccaagcacaaggtggaggagcagccgcctcagccaaagtcagcggcacccgcttcgtgatcgacggcaaaaccggctactttgcaggaacaaactcctactggattggcttcctgaccaacaacagagatgtcgacacaaccttggaccacatcgcctcctcgggcctcaaaatcctccgcgtctggggcttcaacgacgtgaacaaccaaccatccggtaacaccgtctggttccaacgcctcgcctcctcaggctcccaaatcaacaccggccccaacggcctccaacgcctcgactacctcgtcagatcagccgaaacccgcggcatcaagctcatcatcgcgctggtcaactactgggatgactttggcggcatgaaagcctacgtcaacgcctttggaggcacaaaagaatcctggtacaccaacgcccgcgctcaggagcagtacaagcgttacatccaggctgtcgtctcgcgatatgtcaacagccccgcaatctttgcgtgggaacttgccaacgagcccaggtgcaaggggtgcaacacgaatgttattttcaactgggcgacgcagatttcagattatatccggagcttggataaggatcatttgatcacccttggggatgaggggtttgggttgccggggcagacgacgtatccgtatcagtatggggaggggaccgacttcgtcaagaatctgcagattaagaatctggactttgggacgtttcatatgtatcctggtcattggggggtgccgacgagttttggtccagggtggattaaggatcatgcggcggcttgcagggcggcggggaagccgtgtttgttggaggagtatgggtatgagagtgataggtgtaatgtgcagaagggctggcagcaggcgtcgagggagctgagcagggatgggatgagtagtgatttgttttggcaatggggcgatcagttgagtactgggcagacacataatgatgggttcacgatttattatggttcttcgttggctacttgcttggttactgaccatgtgagggctatcaatgctctcccggcg

SEQ ID NO: 16 - DNA, 1068 bp, Artificial Sequence
  mutant (Podospora anserina)
ctcccccaagcacaaggtggaggagcagccgcctcagccaaagtcagcggcacccgcttcgtgatcgacggcaaaaccggctactttgcaggaacaaactcctactggattggcttcctgaccaacaacagagatgtcgacacaaccttggaccacatcgcctcctcgggcctcaaaatcctccgcgtctggggcttcaacgacgtgaacaaccaaccatccggtaacaccgtctggttccaacgcctcgcctcctcaggctcccaaatcaacaccggccccaacggcctccaacgcctcgactacctcgtcagatcagccgaaacccgcggcatcaagctcatcatcgcgctggtcaactactgggatgactttggcggcatgaaagcctacgtcaacgcctttggaggcacaagagaatcctggtacaccaacgcccgcgctcaggagcagtacaagcgttacatccaggctgtcgtctcgcgacatgtcaacagccccgcaatctttgcgtgggaacttgccaacgagcccaggtgcaaggggtgcaacacgaatgttattttcaactgggcgacgcagatttcagattatatccggagcttggataaggatcatttgatcacccttggggatgaggggtttgggttgccggggcagacgacgtatccgtatcagtatggggaggggaccgacttcgtcaagaatctgcagattaagaatctggactttgggacgtttcatatgtatcctggtcattggggggtgccgacgagttttggtccagggtggattaaggatcatgcggcggcttgcagggcggcggggaagccgtgtttgttggaggagtatgggtatgagagtgataggtgtaatgtgcagaagggctggcagcaggcgtcgagggagctgagcagggatgggatgagtggtgatttgttttggcaatggggcgatcagttgagtactgggcagacacataatgatgggttcacgatttattatggttcttcgttggctacttgcttggttactgaccatgtgagggctatcaatgctctcccggcg

SEQ ID NO: 17 - DNA, 1068 bp, Artificial Sequence
  mutant (Podospora anserina)
ctcccccaagcacaaggtggaggagcagccgcctcagccaaagtcagcggcacccgcttcgtgatcgacggcaaaaccggctactttgcaggaacaaactcctactggattggcttcctgaccaacaacagagatgtcgacacaaccttggaccacatcgcctcctcgggcctcaaaatcctccgcgtctggggcttcaacgacgtgaacaaccaaccatccggtaacaccgtctggttccaacgcctcgcctcctcaggctcccaaatcaacaccggccccaacggcctccaacgcctcgactacctcgtcagatcagccgaaacccgcggcatcaagctcatcatcgcgctggtcaactactgggatgactttggcggcatgaaagcctacgtcaacgcctttggaggcacaaaagaatcctggtacaccaacgcccgcgctcaggagcagtacaagcgttacatccaggctgtcgtctcgcgatatgtcaacagccccgcaatctttgcgtgggaacttgccaacgagcccaggtgcaaggggtgcaacacgaatgttattttcaactgggcgacgcagatttcagattatatccggagcttggataaggatcatttgatcacccttggggatgaggggtttgggttgccggggcagacgacgtatccgtatcagtatggggaggggaccgacttcgtcaagaatctgcagattaagaatctggactttgggacgtttcatatgtatcctggtcattgggggttgccgacgagttttggtccagggtggattaaggatcatgcggcggcttgcagggcggcggtgaagccgtgtttgttggaggagtatgggtatgagagtgataggtgtaatgtgcagaagggctggcagcaggcgtcgagggagctgagcagggatgggatgagtggtgatttgttttggcactggggcgatcagttgagtactgggcagacacataatgatgggttcacgatttattatggttcttcgttggctacttgcttggttactgaccatgtgagggctatcaatgctctcccggcg

SEQ ID NO: 18 - DNA, 1068 bp, Artificial Sequence
  mutant (Podospora anserina)
ctcccccaagcacaaggtggaggagcagccgcctcagccaaagtcagcggcacccgcttcgtgatcgacggcaaaaccggctactttgcaggaacaaactcctacaggattggcttcctgaccaacaacagagatgtcgacacaaccttggaccacatcgcctcctcgggcctcaaaatcctccgcgtctggggcttcaacgacgtgaacaaccaaccatccggtaacaccgtctggttccaacgcctcgcctcctcaggctcccaaatcaacaccggccccaacggcctccaacgcctcgactacctcgtcagatcagccgaaacccgcggcatcaagctcatcatcgcgctggtcaactactgggatgactttggcggcatgaaagcctacgtcaacgcctttggaggcacaaaagaatcctggtacaccaacgcccgcgctcaggagcagtacaagcgttacatccaggctgtcgtctcgcgatatgtcaacagccccgcaatctttgcgtgggaacttgccaacgagcccaggtgcaaggggtgcaacacgaatgttattttcaactgggcgacgcagatctcagattatatccggagcttggataaggatcatttgatcacccttggggatgaggggtttgggttgccggggcagacgacgtatccgtatcagtatggggaggggaccgacttcgtcaagaatctgcagattaagaatctggactttgggacgtttcatatgtatcctggtcattggggggcgccgacgagttttggtccagggtggattaaggatcatgcggcggcttgcagggcggcggggaagccgtgtttgttggaggagtatgggtatgagagtgataggtgtaatgtgcagaagggctggcagcaggcgtcgagggagctgagcagggatgggatgagtggtgatttgttttggcaatggggcgatcagttgagtactgggcagacacataatgatgggttcacgatttattatggttcttcgttggctacttgcttggttactgaccatgtgagggctatcaatgctctcccggcg

SEQ ID NO: 19 - DNA, 1347 bp, Artificial Sequence
  mutant (Podospora anserina)
aagccttgcaagcctcgtgatggccccgtgacctacgaggccgaagatgccatcctcaccggcaccaccgtcgacactgctcaggtaggctataccggccgtggctacgtcaccggcttcgacgagggctccgacaagatcaccttccagatcagctccgccaccaccaagctctacgacctctccatccggtacgccgccatctacggtgacaagcgaaccaacgtcgtcctcaacaacggtgccgtcagcgaggtcttcttccccgccggtgactcttttacttctgtcgccgccggccaggtcctcctcaacgctggacaaaacaccatcgacatcgtcaacaactggggatggtacctcatcgactccatcaccctcaccccctccgcccctcgccccccccacgacatcaaccccaacctcaataaccccaacgccgacaccaacgccaagaagctctactcctacctccgctctgtctacggcaacaagatcatctctggccagcaggagctccaccacgccgagtggatcagacagcaaaccggcaagactcccgcgctggtggctgtcgatctgatggattactccccctcccgcgtcgagcgtggcaccaccagccatgccgtcgaggacgccatcgcccaccacaacgcaggcggtatcgtttctgtcctctggcactggaacgctcccgtcggtctgtatgacaccgaagagaacaagtggtggtccggcttctacactcgggctaccgactttgacattgccgccacgttggccaacccccagggtgcgaactacactcttctcatcagggacattgacgcgattgctgtccagctcaagaggctggaggctgctggtgttccggtcttgtggagacctcttcacgaggcggagggtggttggttctggtggggagccaaggggccagagccggcgaagcagctttgggatatcttgtatgagcgtctgacggtgcaccatggtttggataatttgatttgggtgtggaattcgattttggaggattggtatccgggtgatgatacggttgatatcttgtcggccgatgtgtatgcgcagggtaatgggcccatgtcgactcagtacaatgagttgatcgccctcggcagggacaagaagatgattgctgctgcagaggttggcgctgcccctttgcccggcttgttgcaggcttaccaggccaactggctgtggtttgctgtctggggtgatgactttatcagcaaccccagctggaacacggttgctgttctcaacgagatctacaacagcgactatgtgttgacgctggatgagattcaggggtggaggagt

SEQ ID NO: 20 - DNA, 28 bp, Artificial Sequence
  primer
tttgaattcctcccccaagcacaaggtg

SEQ ID NO: 21 - DNA, 31 bp, Artificial Sequence
  primer
ttttctagacccgccgggagagcattgatag

SEQ ID NO: 22 - DNA, 29 bp, Artificial Sequence
  primer
tttatcgataaagccttgtaagcctcgtg

SEQ ID NO: 23 - DNA, 32 bp, Artificial Sequence
  primer
ttttctagaccactcctccacccctgaatctc